(19) United States
(12) Patent Application Publication
(10) Pub. No.: US 2005/0251230 A1
(43) Pub. Date: Nov. 10, 2005

(54) Apparatus and methods for performing phototherapy, photodynamic therapy and diagnosis

(76) Inventors: Nicholas B. MacKinnon, Vancouver (CA); Ulrich Stange, Vancouver (CA)

Correspondence Address: GRAYBEAL, JACKSON, HALEY LLP, 155 - 108TH AVENUE NE, SUITE 350, BELLEVUE, WA, 98004-5901, US

(21) Appl. No.: 10/951,449

(22) Filed: Sep. 27, 2004

Related U.S. Application Data
(60) Provisional application No. 60/506,280, filed on Sep. 26, 2003.

(51) Int. Cl. A61N 5/06

(52) U.S. Cl. 607/88; 607/92

(57) ABSTRACT

Computer-controlled illumination systems that can be used to select a variety of wavelengths of light and intensities of such wavelengths suitable for the activation of various kinds of photodynamic drugs, for various types of phototherapy. If desired, the systems can work interactively with a measurement system to measure the quantity of some types of photodynamic drugs present in a tissue.

### CROSS-REFERENCE TO RELATED APPLICATIONS

The present application claims priority from pending U.S. provisional patent application No. 60/506,280 filed Sep. 26, 2003.

### BACKGROUND

There are many types of therapeutic interventions than can be used to treat illness, disease, disorders and or concerns about appearance. These interventions can include surgery, pharmaceuticals, physical manipulation such as massage or physiotherapy, topical creams, acupuncture and other therapies. An increasingly used form of therapeutic intervention is the use of light, both for the diagnosis and treatment of disease.

One form of therapeutic intervention with light is called phototherapy. Phototherapy is the illumination of tissue with light to induce some form of therapeutic effect or healing. Light is well known to interact with tissues and other materials at a molecular level. A number of techniques exploiting this property have been developed. Examples of these are the treatment of psoriasis or other skin conditions with ultraviolet light, the use of blue light to break down excess bilirubin in infants with hyperbilirubinemia, sometimes called jaundice, and the use of red light to speed wound healing.

Another form of therapeutic intervention with light is photodynamic therapy. Photodynamic therapy is based on the introduction of a drug, either systemically by injection or intravenous drip, oral ingestion, or topical application either directly or by breathing in the drug as a nebulized mixture. The action of the drug does not take effect until it is triggered by the presence of light of a particular energy and intensity When sufficient drug has been administered to the area of the body to be treated, the drug can be activated at the desired area by illuminating the tissue. Thus the effect of the drug can be substantially limited to the desired area of treatment. Photodynamic therapy is an established and approved form of therapy for a number of conditions, including cancer, macular disease, skin conditions and other problems. New forms of photodynamic therapy are continuously being developed, from treatments for baldness to infection control.

For every type of photodynamic drug there is a characteristic wavelength or range of wavelengths of light that can be used to trigger the drug's activity. Often these wavelengths of light are very specific. This specificity is what helps prevent them being activated in a way that is not desirable, or at a time that is not desirable.

Often photodynamic drugs have a time dependent response. The drugs will accumulate in a desired tissue either by some from of preferential accumulation or by some form of delayed clearing from the tissue. Thus the application of light must often be well controlled for intensity and duration as well as wavelength for the therapy to be effective.

Some photodynamic drugs also have optical properties, such as fluorescence (or other emitted light) in addition to their therapeutic effect. These additional optical properties can be used for optical measurements to measure the amount of drug in the tissue, and to measure how much of the drug has been consumed after treatment light has been applied.

Most photodynamic drugs are provided with particular instruments to provide the illumination light to trigger the therapy. Lasers are often used because they provide a narrow wavelength range with sufficient power for activation and can be coupled into fibers.

Sometimes filtered white light sources such as xenon arc lamps or other sources are used to provide the illumination. These sources employ narrow band filters to limit the light to only desired wavelengths. Such narrow band filters mean much of the light from the light source must be absorbed by the filter to prevent the undesired wavelengths from reaching the tissue, resulting in thermal stresses and the need for cooling strategies that add to the cost of the equipment.

A problem with many of these light sources is that they are only suitable for one type of drug or one type of therapy. This requires a medical facility to purchase and maintain many types of light sources, which can be costly.

Thus there has gone unmet a need for a light source that can be used to activate a range of drugs, that can be well controlled for duration and intensity of exposure when activating a drug, and can further be used if desired to measure the presence of a photodynamic drug.

### SUMMARY

The apparatus and methods, etc., herein provide a computer-controlled illumination system that can be used to select a variety of wavelengths of light suitable for the activation of various kinds of photodynamic drugs, for various types of phototherapy, and, if desired, that can work interactively with a measurement system to measure the quantity of some types of photodynamic drugs present in a tissue.

The computer-controlled illumination system comprises a tunable light source that comprises a source of light, a spectrum former such as a prism or diffraction grating and a pixelated spatial light modulator (pixelated SLM) (RPSLM) such as a digital micromirror device or liquid crystal on silicon (LCOS), or other suitable tunable light filter such as a transmissive pixelated spatial light modulator, or acousto-optic tunable filter (AOTF). The light from the light source is directed as a beam to the wavelength dispersive element which disperses the beam into a spectrum that is imaged onto a RPSLM. The pixel elements of the RPSLM can be switched to select wavelengths of light and selected amounts of the selected wavelengths of light to propagate. The light source can also comprise a plurality of different light emanators, for example to provide greater total intensity or each providing a different wavelength or wavelength band(s) of light in combination with a selective device(s) configured to transmit desired amounts of the different wavelength band(s). Exemplary light sources include red, green and blue LEDs or other desired lamps and photon generators, and exemplary selective devices include rheostats that control the power and thus output of the light sources, as well as various other wavelength and intensity selective elements discussed herein. The light that propagates is then, if desired, optically mixed together and directed to the illumination path, for example of an endoscope or other medical device.

The SLM may be operably connected to a controller, which controller contains computer-implemented programming that controls the on/off pattern of the pixels in the SLM. The controller can be located in any desired location to the rest of the system. For example, the controller can be either within a housing of the source of illumination or it can be located remotely, connected by a wire, cellular link or radio link to the rest of the system. If desired, the controller, which is typically a single computer but can be a plurality of linked computers, a plurality of unlinked computers, computer chips separate from a full computer or other suitable controller devices, can also contain one or more computer-implemented programs that provide specific lighting characteristics, i.e., specific desired, selected spectral outputs and wavelength dependent intensities, corresponding to known wavelength bands that are suitable for or a specific light for disease diagnosis or treatment, or to invoke disease treatment (for example by activating a drug injected into a tumor in an inactive form), or other particular situations.

In one aspect, the present apparatus and methods provides a computer-controlled illumination system that provides a variable selected spectral output and a variable wavelength dependent intensity distribution. The system comprises a) a spectrum former able to provide a spectrum from light generated by the source of light, and b) a reflective pixelated spatial light modulator (RPSLM) located downstream from and optically connected to the spectrum former, the RPSLM reflecting substantially all light impinging on the RPSLM and switchable to reflect light from the spectrum former between at least first and second reflected light paths. Typically, at least one or both of the light paths that do not reflect back to the spectrum former. The RPSLM can be a digital micromirror device. The RPSLM is operably connected to at least one controller containing computer-implemented programming that controls an on/off pattern of pixels in the RPSLM to reflect a desired segment of light in the spectrum to the first reflected light path and reflect substantially all other light in the spectrum impinging on the RPSLM to another light path, the desired segment of light consisting essentially of a desired selected spectral output and a desired wavelength dependent intensity distribution.

In some embodiments, the spectrum former comprises at least one of a prism and a diffraction grating, which can be a reflective diffraction grating, transmission diffraction grating, variable wavelength optical filter, or a mosaic optical filter. The system may or may not comprise, between the spectrum former and the SLM, an enhancing optical element that provides a substantially enhanced image of the spectrum from the spectrum former to the SLM. The SLM can be a first SLM, and the desired segment of light can be directed to a second SLM operably connected to the same controller or another controller containing computer-implemented programming that controls an on/off pattern of pixels in the second SLM to reflect the desired segment or other segment of light in one direction and reflect other light in the spectrum in at least one other direction. The system can further comprise an optical projection device located downstream from the first SLM to project light out of the lighting system as a directed light beam.

The illumination light can be selected to substantially mimic a spectral output and a wavelength dependent intensity distribution of at least one of the output energy for disease treatment, photodynamic therapy, or drug dosimetry.

The computer-controlled illumination system can further comprise a sensor optically connected to and downstream from the SLM, the sensor also operably connected to a controller containing computer-implemented programming able to determine from the sensor whether the desired segment contains a desired selected spectral output and a desired wavelength dependent intensity distribution, and adjust the on/off pattern of pixels in the pixelated SLM to improve the correspondence between the desired segment and the desired selected spectral output and the desired wavelength dependent intensity distribution. The system can also comprise a heat management system operably connected to the tunable light source to remove undesired energy emitted from the tunable light source toward at least one of the SLM, and the spectrum former.

The heat management system can be located between the spectrum former and the pixelated SLM and the spectrum former, or elsewhere as desired. The heat management system can comprise a dichroic mirror. The dichroic mirror can transmit desired wavelengths of light and reflect undesired wavelengths of light, or vice-versa. The undesired energy can be directed to an energy absorbing surface and thermally conducted to a radiator. The heat management system can be an optical cell containing a liquid that absorbs undesired wavelengths and transmits desired wavelengths. The liquid can be substantially water and can flow through the optical cell via an inlet port and outlet port in a recirculating path between the optical cell and a reservoir. The recirculating path and the reservoir can comprise a cooling device, which can be a refrigeration unit, a thermoelectric cooler, or a heat exchanger.

The computer-controlled illumination system can further comprise a spectral recombiner optically connected to and located downstream from the pixelated spatial light modulator, which can comprise a prism, a Lambertian optical diffusing element, a directional light diffuser such as a holographic optical diffusing element, a lenslet array, or a rectangular light pipe. In one embodiment, the spectral recombiner can comprise an operable combination of a light pipe and at least one of a lenslet array and a holographic optical diffusing element. The detector can be located in the at least one other direction, and can comprise at least one of a CCD, a CID, a CMOS, and a photodiode array. The source of light, the spectrum former, the enhancing optical element that provides an enhanced image, the SLM, and the projection system, can all be located in a single housing, or fewer or more elements can be located in a single housing.

In another aspect of the apparatus and methods the computer-controlled illumination system or an endoscopy system comprises an adapter or other apparatus for mechanically and/or optically connecting the illumination light guide of an endoscope to the output of the computer-controlled illumination system. The illumination light guide of the endoscope can be at least one of an optical fiber, optical fiber bundle, liquid light guide, hollow reflective light guide, or free-space optical connector. The light guide may be integral with the endoscope or it may be modular and separable from the endoscope.

In some embodiments of the apparatus and methods the endoscope system can comprise an image detector, which can be an unfiltered image sensor. An unfiltered image sensor relies on the natural optical response of the sensor material to light impinging on the sensor to generate an image data.

In other embodiments of the apparatus and methods the image detector can have an optical filter placed in front of it to limit the wavelengths of light that reach the detector. It may also have a matrix filter that only allows selected wavelengths to reach selected pixels. The optical filter can be at least one of a long-pass filter, a short-pass filter, a band-pass filter, or a band-blocking filter. The matrix optical filter can be at least two of a long-pass filter, a short-pass filter, a band-pass filter, or a band-blocking filter. A long-pass filter is useful to block undesired wavelengths such as ultraviolet light or fluorescence excitation light from impinging on the sensor. A short-pass filter is useful to block undesired wavelengths such as infrared light from impinging on the sensor. A band-pass filter may be useful to allow only selected wavelengths such as visible light to impinge on the detector. A band-blocking filter is useful to block fluorescence excitation light from impinging on the image sensor.

In some embodiments of the apparatus and methods, the image detector can be operably connected to the controller and synchronized to the computer-controlled illumination system to provide sequences of images of tissue illuminated by desired wavelengths of light and captured as images. These images can then be combined or processed as desired to provide useful information to the physician or surgeon.

In another embodiment of the apparatus and methods, the image detector can be synchronized to the computer-controlled illumination system to provide sequences of images of tissue illuminated by desired wavelengths of light and captured as images. These images can then be combined or processed as desired to provide useful information to the physician or surgeon.

The endoscope system or other medical optical system can further comprise computer controlled image acquisition and processing systems that can analyze the information from an image or sequence of images and present it in a way that is meaningful to an operator.

The computer-controlled illumination system and image detector may be operably connected to a controller, which controller contains computer-implemented programming that controls the time of image acquisition in the image detector and the wavelength distribution and duration of illumination light from the computer-controlled illumination system. The controller can be located in any desired location relative to the rest of the system. For example, the controller can be either within a housing of the source of illumination or it can be located remotely, connected by a wire, cellular link or radio link to the rest of the system. If desired, the controller, which is typically a single computer but can be a plurality of linked computers, a plurality of unlinked computers, computer chips separate from a full computer or other suitable controller devices, can also contain one or more computer-implemented programs that provide control of image acquisition and/or control of specific lighting characteristics, i.e., specific desired, selected spectral outputs and wavelength dependent intensities, corresponding to known wavelength bands that are suitable for imaging or a specific light for disease diagnosis or treatment, or to invoke disease treatment (for example by activating a drug injected into a tumor in an inactive form), or other particular situations.

In a further aspect, the present apparatus and methods provides methods of illuminating a tissue comprising: a) generating an illumination light containing a desired spectral output and wavelength dependent intensity distribution from a computer-controlled illumination system; b) sensing the illumination light with a sensor; and directing the illumination light toward a tissue.

The methods of illuminating a tissue can further comprise: a) directing a light beam along a light path and through a spectrum former to provide a spectrum from the light beam traveling; and, b) passing the spectrum via a pixelated spatial light modulator located downstream from and optically connected to the spectrum former, the pixelated spatial light modulator operably connected to at least one controller containing computer-implemented programming that controls an on/off pattern of pixels in the pixelated spatial light modulator, wherein the on/off pattern can be set to pass a desired segment of light in the spectrum in one direction and interrupt other light in the spectrum impinging on the pixelated spatial light modulator, to provide illumination light consisting essentially of a selected spectral output and a selected wavelength dependent intensity distribution.

The methods further can comprise emitting the light beam from a light source located in a same housing as and upstream from the spectrum former. The methods further can comprise switching the modified light beam between the first reflected light path and the second reflected light path. The methods further can comprise passing the light beam by an enhancing optical element between the spectrum former and the pixelated SLM to provide a substantially enhanced image of the spectrum from the spectrum former to the pixelated SLM. The pixelated SLM can be a first reflective pixelated spatial light modulator, and the methods further can comprise reflecting the modified light beam off a second pixelated SLM operably connected to at least one controller containing computer-implemented programming that controls an on/off pattern of pixels in the second RPSLM to reflect the desired segment of light in one direction and reflect other light in the spectrum in at least one other direction.

The methods further can comprise passing the modified light beam by an optical projection device located downstream from at least one of the first pixelated SLM and the second pixelated SLM to project illumination light.

The methods can further comprise diverting a portion of the illumination light to a sensor optically connected to and downstream from the SLM, the sensor can be operably connected to the controller, wherein the controller contains computer-implemented programming able to determine from the detector whether the desired segment contains the desired selected spectral output and the desired wavelength dependent intensity distribution, exist in the illumination light. The methods can comprise adjusting the on/off pattern of pixels in the SLM to obtain or maintain the desired selected spectral output and the desired wavelength dependent intensity distribution of the illumination light.

The methods can also comprise removing undesired energy emitted from the light source toward at least one of the pixelated SLM and the spectrum former, the removing effected via a heat management system operably connected to the tunable light source. The methods further can comprise a spectral recombiner optically connected to and located downstream from the pixelated SLM.

The methods can further comprise directing the illumination light toward a tissue by at least one of directly illuminating the tissue via projection, or directing the illumination light into the light guide of an endoscope, or directing the illumination light into the light guide of a surgical microscope or other imaging system for viewing tissue, or directing the illumination light into a light guide such as an optical fiber or a bundle of optical fibers, or into a light guide fitted with an optical diffusing or directing element at the distal end of the fiber, proximal to the tissue.

The methods can further comprise selecting at least one of a desired wavelength range suitable for activating a drug used for photodynamic therapy, a desired intensity of illumination suitable for activating a drug for photodynamic therapy, a desired duration of illumination suitable for activating a drug for photodynamic therapy.

The methods can further comprise selecting at least one of a desired wavelength range suitable for phototherapy, a desired intensity of illumination suitable for phototherapy, a desired duration of illumination suitable phototherapy.

The methods can further comprise selecting at least one of a desired wavelength range suitable for measuring the amount of a photodynamic drug present in tissue, a desired intensity of illumination suitable for measuring the amount of a photodynamic drug present in tissue, a desired duration of illumination suitable measuring the amount of a photodynamic drug present in tissue, and a measuring device such as a spectrometer or an optical imaging system.

The methods can further comprise measuring the amount of a photodynamic drug, where the method of measuring a drug comprises at least one of measuring the optical reflectance spectral characteristics, optical fluorescence (or other emitted light) characteristics, single, dual or multispectral reflectance imaging, single, dual or multispectral fluorescence imaging.

The methods can further comprise illuminating the tissue with a sequence of different kinds of illumination that can alternately provide illumination suitable for activating a therapy, and illumination for measuring the progress of the therapy.

These and other aspects, features and embodiments are set forth within this application, including the following Detailed Description and attached drawings. The discussion herein provides a variety of aspects, features, and embodiments; such multiple aspects, features and embodiments can be combined and permuted in any desired manner. In addition, various references are set forth herein that discuss certain apparatus, systems, methods, or other information; all such references are incorporated herein by reference in their entirety and for all their teachings and disclosures, regardless of where the references may appear in this application. Such incorporated references include: U.S. Pat. No. 6,781,691; pending U.S. patent application Ser. No. 10/893,132, entitled Apparatus And Methods Relating To Concentration And Shaping Of Illumination, filed Jul. 16, 2004; pending U.S. patent application No. ______ (attorney docket no. 1802-9-3), entitled Apparatus And Methods Relating To Color Imaging Endoscope Systems, filed contemporaneously herewith; pending U.S. patent application No. ______ (attorney docket no. 1802-12-3), entitled Apparatus And Methods Relating To Precision Control Of Illumination Exposure, filed contemporaneously herewith; pending U.S. patent application No. ______ (attorney docket no. 1802-13-3), entitled Apparatus And Methods Relating To Expanded Dynamic Range Imaging Endoscope Systems, filed contemporaneously herewith; pending U.S. patent application No. ______ (attorney docket no. 1802-15-3), entitled Apparatus And Methods Relating To Enhanced Spectral Measurement Systems, filed contemporaneously herewith.

### BRIEF DESCRIPTION OF THE DRAWINGS

FIG. 1 provides a schematic depiction of a computer-controlled illumination system according to an embodiment of the invention.

FIG. 2A provides a schematic depiction of the broadband spectrum of illumination light that may be emitted from the computer-controlled illumination system in FIG. 1.

FIG. 2B provides a schematic depiction of a selected spectrum of illumination light that is selected from the broadband spectrum in FIG. 2A to provide a wavelength dependent intensity distribution suitable for phototherapy and/or photodynamic therapy, according to an embodiment of the invention.

FIG. 3A provides a schematic depiction of the broadband spectrum of illumination light that may be emitted from the computer-controlled illumination system in FIG. 1.

FIG. 3B provides a schematic depiction of a selected spectra of illumination light that are selected from the broadband spectrum in FIG. 3A to provide a wavelength dependent intensity distribution suitable for photodynamic therapy and for measuring the quantity of a photodynamic drug remaining in a tissue, according to an embodiment of the invention.

FIG. 4 provides a schematic depiction of the selected spectra of illumination light in FIG. 3B that the computer-controlled illumination system in FIG. 1 emits sequentially over time, according to an embodiment of the invention.

FIG. 5 provides a schematic depiction of selected different exposure intensities and durations of a selected spectrum of illumination light that the computer-controlled illumination system in FIG. 1 may emit for phototherapy, photodynamic therapy or measurement, according to an embodiment of the invention.

FIG. 6A provides a schematic depiction of an endoscopy system comprising the computer-controlled illumination system in FIG. 1, according to an embodiment of the invention.

FIG. 6B provides a schematic depiction of a partial view of a distal end of the endoscopy system in FIG. 6A.

FIG. 7 is a flow chart depicting a power management scheme according to the present invention.

### DETAILED DESCRIPTION

The present apparatus and methods comprise a computer-controlled illumination system that one may use to generate light for therapeutic intervention. For example, the computer-controlled illumination system may be used for phototherapy in which one or more tissues such as skin, muscle and internal organs, etc. are illuminated with light, or photodynamic therapy in which a drug or some other chemical is introduced into one or more tissues and activated by light, or diagnosis in which the presence of a drug or some other chemical in one or more tissues is revealed. With the computer-controlled illumination system, one may selectively generate light that has a specific spectral output and a specific wavelength dependent intensity distribution for phototherapy, photodynamic therapy and diagnosis. Furthermore, the spectral output and wavelength dependent intensity distribution of the light generated by the computer illumination system may be varied to correspond with different phototherapies, photodynamic therapies and diagnosis, or changing conditions within a phototherapeutic procedure, photodynamic procedure and a diagnostic procedure.

Turning to some general information about light, the energy distribution of light is what determines the nature of its interaction with an object, compound or organism. A common way to determine the energy distribution of light is to measure the amount or intensity of light at various wavelengths to determine the energy distribution or spectrum of the light. To make light from a light source useful for a particular purpose it can be conditioned to remove undesirable wavelengths or intensities, or to enhance the relative amount of desirable wavelengths or intensities of light. For example, a high signal-to-noise ratio and high out-of-band rejection enhances the spectral characteristics of light.

The systems and methods, including kits and the like comprising the systems or for making or implementing the systems or methods, provide the ability to selectively, and variably, decide which colors, or wavelengths, of light will be projected from the system, and how strong each of the wavelengths will be. The wavelengths can be a single wavelength, a single band of wavelengths, a group of wavelengths/wavelength bands, or all the wavelengths in a light beam. If the light comprises a group of wavelengths/wavelengths bands, the group can be either continuous or discontinuous. The wavelengths can be attenuated so that the relative level of one wavelength to another can be increased or decreased (e.g., decreasing the intensity of one wavelength among a group of wavelengths effectively increases the other wavelengths relative to the decreased wavelength). This is advantageous because such fine control of spectral output and wavelength dependant intensity distribution permits a single illumination system to provide highly specialized light for phototherapy, photodynamic therapy or diagnosis.

### DEFINITIONS

The following paragraphs provide definitions of some of the terms used herein. All terms used herein, including those specifically described below in this section, are used in accordance with their ordinary meanings unless the context or definition indicates otherwise. Also unless indicated otherwise, except within the claims, the use of “or” includes “and” and vice-versa. Non-limiting terms are not to be construed as limiting unless expressly stated (for example, “including” and “comprising” mean “including without limitation” unless expressly stated otherwise).

A “controller” is a device that is capable of controlling a spatial light modulator, a detector or other elements of the apparatus and methods herein. A “controller” contains or is linked to computer-implemented programming. Typically, a controller comprises one or more computers or other devices comprising a central processing unit (CPU) and directs other devices to perform certain functions or actions, such as the on/off pattern of the pixels in the pixelated SLM, the on/off status of pixels of a pixelated light detector (such as a charge coupled device (CCD) or charge injection device (CID)), and/or compile data obtained from the detector, including using such data to make or reconstruct images or as feedback to control an upstream spatial light modulator. A computer comprises an electronic device that can store coded data and can be set or programmed to perform mathematical or logical operations at high speed. Controllers are well known and selection of a desirable controller for a particular aspect of the present apparatus and methods is within the scope of the art in view of the present disclosure.

A “spatial light modulator” (SLM) is a device that is able to selectively modulate light. The present apparatus and methods comprise one or more spatial light modulators disposed in the light path of an illumination system. A pixelated spatial light modulator comprises an array of individual pixels, which are a plurality of spots that have light passing characteristics such that they transmit, reflect or otherwise send light along a light path, or instead block the light and prevent it or interrupt it from continuing along the light path. Such pixelated arrays are well known, having also been referred to as a multiple pattern aperture array, and can be formed by an array of ferroelectric liquid crystal devices, electrophoretic displays, or by electrostatic microshutters. See, U.S. Pat. Nos. 5,587,832; 5,121,239; R. Vuelleumier, Novel Electromechanical Microshutter Display Device, Proc. Eurodisplay '84, Display Research Conference September 1984.

A reflective pixelated SLM comprises an array of highly reflective mirrors that are switchable between at least an on and off state, for example between at least two different angles of reflection or between present and not-present. Examples of reflective pixelated SLMs include digital micromirror devices (DMDs), liquid crystal on silicon (LCOS) devices, http://www.intel.com/design/celect/technology/lcos/, as well as other MicroElectroMechanical Structures (MEMS). DMDs can be obtained from Texas Instruments, Inc., Dallas, Tex., U.S.A. In the DMD embodiment, the mirrors have three states. In a parked or “0” state, the mirrors parallel the plane of the array, reflecting orthogonal light straight back from the array. In one energized state, or a “−10” state, the mirrors fix at −10° relative to the plane of the array. In a second energized state, or a “+10” state, the mirrors fix at +10° relative to the plane of the array. Other angles of displacement are possible and are available in different models of this device. When a mirror is in the “on” position light that strikes that mirror is directed into the illumination light path. When the mirror is in the “off” position light is directed away from the illumination light path. On and off can be selected to correspond to energized or non-energized states, or on and off can be selected to correspond to different energized states. If desired, the light directed away from the projection light path can also be collected and used for any desired purpose (in other words, the DMD can simultaneously or serially provide two or more useful light paths). The pattern in the DMD can be configured to produce two or more spectral and intensity distributions simultaneously or serially, and different portions of the DMD can be used to project or image along two or more different projection light paths.

An “illumination light path” is the light path from a light source to a target tissue or scene, while a “detection light path” is the light path for light emanating to a detector. The light includes ultraviolet (UV) light, blue light, visible light, near-infrared (NIR) light and infrared (IR) light.

“Upstream” and “downstream” are used in their traditional sense wherein upstream indicates that a given device is closer to a light source, while downstream indicates that a given object is farther away from a light source.

The scope of the present apparatus and methods includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

Other terms and phrases in this application are defined in accordance with the above definitions, and in other portions of this application.

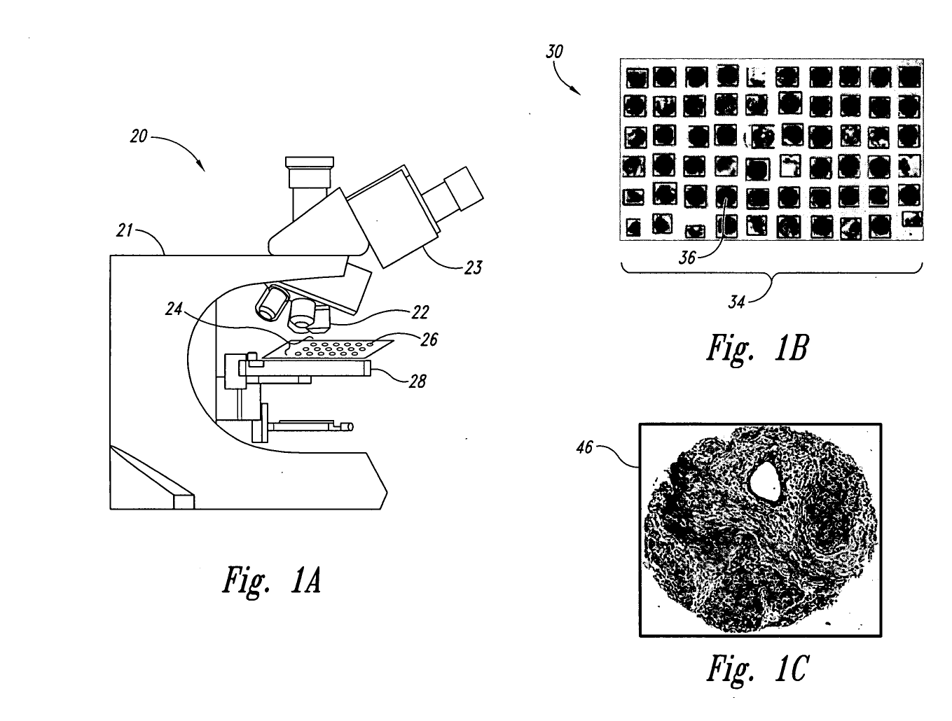

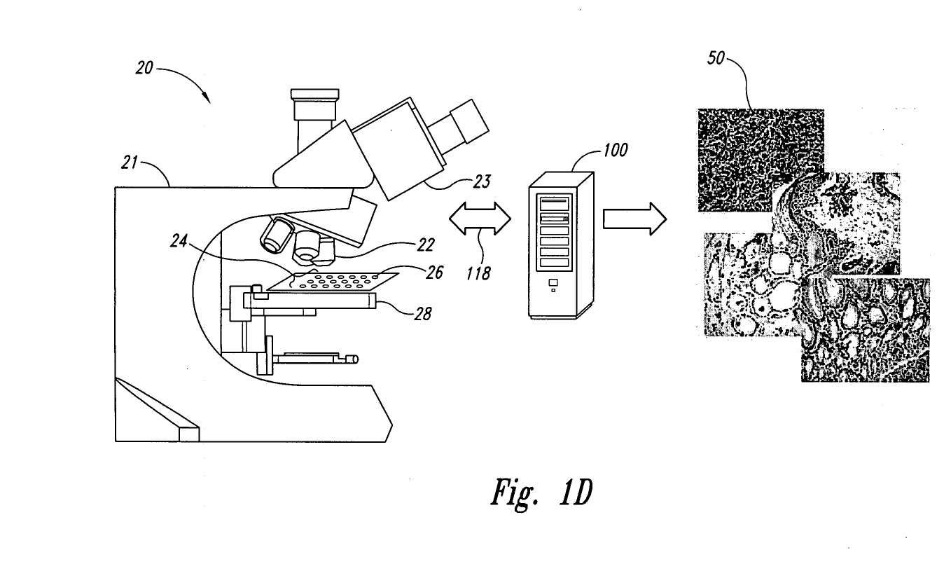

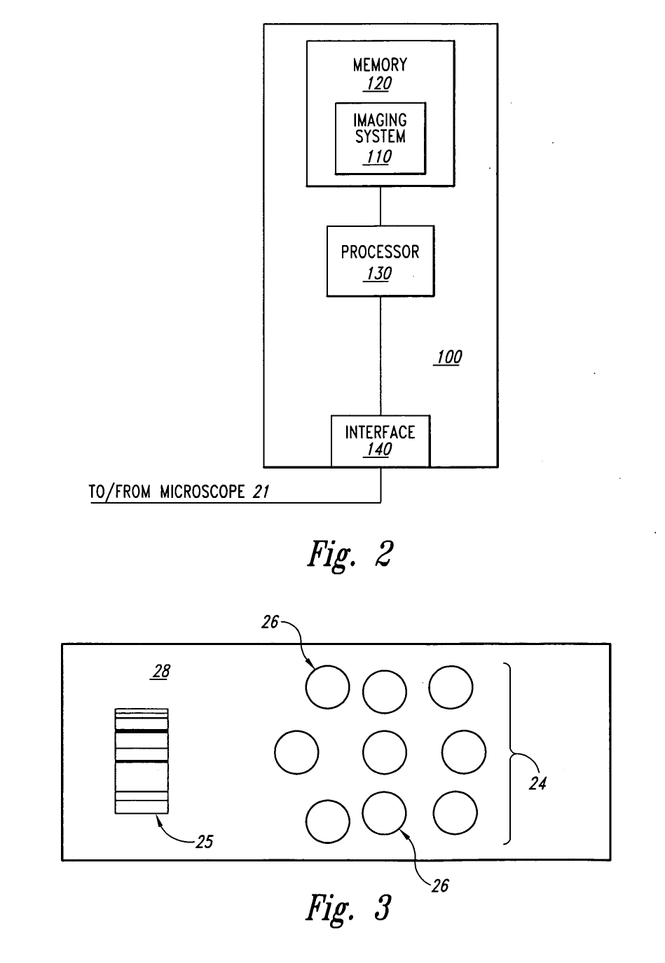

FIG. 1 provides a schematic depiction of a computer-controlled illumination system 10 according to an embodiment of the invention. The computer-controlled illumination system 10 generates and emits an illumination light 12 having a selected spectral output and a selected wavelength dependent intensity distribution that may be directed to tissue 14 for at least one of the following: phototherapeutic procedures, photodynamic procedures and diagnostic procedures (discussed in greater detail in conjunction with FIGS. 2A-5). Furthermore, one may easily vary the spectral output and a selected wavelength dependent intensity distribution of the illumination light 12 as desired to correspond with different procedures or different conditions within the same procedure (also discussed in greater detail in conjunction with FIGS. 2A-5). The computer-controlled illumination system 10 as shown comprises a tunable light source 16 for generating and emitting the illumination light 12, a sensor 18 for detecting the illumination light 12 and transmitting data representing the spectral output and wavelength depended intensity distribution of the illumination light 12, and a controller 20 for coordinating the tunable light source 16 and sensor 18 to provide a desired illumination light 12.

The tunable light source 16 provides virtually any desired color(s) and intensity(s) of light, from white light, or light that is visible to an unaided human eye, to light containing only a certain color(s) and intensity(s). The colors, or “spectral output,” which means a particular wavelength, band of wavelengths, or set of wavelengths, as well as the intensities, which means a “wavelength dependent intensity distribution,” can be combined and varied as desired. The tunable light source may also provide other kinds of light, such as UV light and infrared light.

The tunable light source 16 comprises a source of light 22 to generate light 24, and a tunable filter 26 to generate a desired spectral output and wavelength dependent intensity distribution. The tunable filter 26 may be any desired device capable of modulating the light 24 from the source of light 22. For example, the tunable filter 26 may comprise a spectrum former 28 to separate the light 24 into its spectral components 30, and a pixelated SLM 32 to combine selected spectral components to generate the illumination light 12 having the desired spectral output and wavelength dependent intensity distribution, and to separate unwanted spectral components 34 from the selected spectral components. By selectively turning on or off individual pixels of the SLM, one can generate illumination light 12 having a desired spectral output and a desired wavelength dependent intensity distribution. For example, only one narrow wavelength of light from the spectral components 30, such as only a pure green line of light in a typical linear spectrum may be generated, or non-linear spectra can be generated. By varying the duty cycle of some of the pixels to be turned on or off, virtually any spectral distribution of light can be created. The pixelated SLM 32 may be transmissive or reflective. In other embodiments, the tunable filter may comprise an acousto-optic tunable filter. Suitable tunable light sources are discussed, e.g., in U.S. Pat. No. 6,781,691 and U.S. patent application Ser. No. 10/893,132.

The sensor 18 transmits the data representing the spectral output and wavelength dependent intensity distribution to the controller 20 and may be any desired device capable of sensing the illumination light 12 and generating data representing the spectral distribution and wavelength dependent intensity distribution of the illumination light 12. For example, the sensor 18 may comprise spectrometers, spectroradiometers, charge coupled devices (CCDs), charge injection devices (CIDs), a complementary metal-oxide semi-conductors (CMOSs), photodiode arrays. In some embodiments, the sensor 18 receives illumination light 12 from a beam splitter such as lens 36 so that the illumination light 12 projected toward the tissue is not affected by the sensor 18.

The controller 20 receives the data representing the spectral output and wavelength dependent intensity distribution from the sensor 18 and includes computer-implemented programming to coordinate the tunable light source, and the sensor. Such coordination typically comprises determining whether the spectral output and wavelength dependent intensity distribution of the illumination light 12 is the selected spectral output and wavelength dependent intensity distribution, and varying the spectral output and/or wavelength dependent intensity distribution of the illumination light 12 as desired. In some embodiments, the controller 20 is operably connected to the SLM 32, and the computer-implemented programming controls the on/off pattern of the pixels. Suitable controllers are discussed, e.g., in U.S. Pat. No. 6,781,691 and U.S. patent application Ser. No. 10/893,132.

The computer-controlled illumination system 10 may include other components as desired. For example, the computer-controlled illumination system 10 may comprise at least one of the following: a projection system (not shown) to project the illumination light 12 toward the tissue 14, and a heat management system (also not shown) to remove undesired energy generated by the tunable light source. The projection system may be desirable to enlarge, decrease or change the geometric form of the coverage area (not shown) of the illumination light 12 on the tissue 14 area and may comprise any desired optical device to accomplish this. For example, the projection system may include lenses and may focus the illumination light onto an area of the tissue 14 that is less than the coverage area would be without the projection system, or the projection system may disperse the illumination light onto an area of the tissue 14 that is more than the coverage area would be without the projection system, and/or the projection system may modify the illumination light 12 to project the illumination light in a form that corresponds to the form of a region of the tissue to be illuminated, such as a long, narrow region corresponding to a skin laceration, or an irregular shaped region such as a cancer lesion, or area affected by a skin ailment such as psoriasis.

The projection system may match the shape and size of the illumination area to correspond to the shape and size of a target region discerned by an imaging system, for example an imaging sensor and image processing system of an endoscope or surgical microscope, such as those discussed herein. For example, in the treatment of a skin lesion such as a skin cancer (e.g., melanoma or basal cell carcinoma) or psoriasis, the imaging system can use standard image analysis techniques to identify cancerous/diseases regions or cells, then use such information to control a spatial light modulator such as an RPSLM, DMD, LCOS, liquid crystal diode, etc., such that the SLM transmits therapeutic, diagnostic, etc., light to the skin. If desired, the cross-sectional shape of the illumination light can be altered during treatment or other usage such that the treatment light is modified on-the-fly to treat the actively modifying shape of the target.

The heat management system may comprise any desired component or assembly of components and may be configured relative to the tunable light source to remove undesired energy emitted from the source of light 22. For example, the heat management system may comprise an energy-absorbing surface, preferably one thermally connected to thermally conduct the heat to a radiator, or an optical cell containing a liquid that absorbs undesired wavelengths and transmits desired wavelengths, such as water. For embodiments where the heat management system comprises an optical cell, the optical cell can also comprise an inlet port and an outlet port so that fresh liquid can be provided, and if desired the liquid can flow in a re-circulating path between the optical cell and a reservoir. The re-circulating path or the reservoir can further comprise a cooling device such as a refrigeration unit, a thermal-electric cooler or a heat exchanger. Suitable projection and heat management systems are discussed, e.g., in U.S. Pat. No. 6,781,691 and U.S. patent application Ser. No. 10/893,132.

Because the computer-controlled illumination system 10 can provide an illumination light 12 having a desired spectral output and wavelength dependent intensity distribution, and can vary the spectral output and wavelength dependent intensity distribution as desired, the computer-controlled illumination system may be easily used for a variety of phototherapy, photodynamic therapy and diagnostic procedures. For example, the computer-controlled illumination system may be used to generate an illumination light 12 for phototherapy, photodynamic therapy and/or diagnosis, that requires a substantially consistent spectral output and wavelength dependent intensity distribution for a period of time. For some therapies and diagnosis, the substantially consistent spectral output and wavelength dependent intensity distribution may comprise two or more portions, each selected to perform a certain function. For example, one portion may comprise a spectral distribution and wavelength dependent intensity distribution for measuring the amount of a drug present in the tissue 14, and another portion may comprise a spectral distribution and wavelength dependent intensity distribution for activating the drug. The computer-controlled illumination system may also be used to generate an illumination light 12 for phototherapy, photodynamic therapy and/or diagnosis, that requires different spectral outputs and wavelength dependent intensity distributions at different times during the therapy or diagnosis. For example, a photodynamic therapy may comprise locating the location of a drug present in the tissue 14 with a certain spectral output and wavelength dependent intensity distribution, and then, activating the drug with another certain spectral output and wavelength dependent intensity distribution. The different spectral outputs and wavelength dependent intensity distributions may form one sequence or they may form a sequence of the sequences, such as repeatedly alternating between two different spectral outputs and wavelength dependent intensity distributions.

FIG. 2A provides a schematic depiction of a broadband spectrum 40 of light that may be emitted from a light source such as in computer-controlled illumination system 10 (FIG. 1). FIG. 2B provides a schematic depiction of a selected spectrum of illumination light 12 (FIG. 1) that is selected from the broadband spectrum 40 to provide a spectrum output and a wavelength dependent intensity distribution suitable for phototherapy, photodynamic therapy or diagnosis, according to an embodiment of the invention.

The broadband spectrum 40 may be generated from any desired source of light 22 (FIG. 1). For example, the broadband spectrum 40 may be generated from a xenon lamp and comprise a spectrum that is visible and appears white to an unaided human eye. The spectrum 42 represents a portion of the broadband spectrum 40 that is suitable for phototherapy, photodynamic therapy or diagnosis. Spectrum 44 represents the spectral output of the illumination light 12 that is generated by the pixelated SLM 32 (FIG. 1). By controlling the on and off pattern of the pixels of the SLM 32 or other light-controlling elements in other SLMs, one can obtain any portion of the broadband spectrum 40 and separate the remaining portions, as desired, to generate an illumination light 12 having a spectral output and wavelength dependent intensity distribution suitable for performing a variety of phototherapeutic procedures, photodynamic therapeutic procedures and diagnostic procedures.

FIG. 3A provides a schematic depiction of the broadband spectrum 46 of light that may be emitted from the computer-controlled illumination system 10 (FIG. 1). FIG. 3B provides a schematic depiction of selected spectra of illumination light 12 (FIG. 1) that are selected from the broadband spectrum 46 to provide a spectrum output and a wavelength dependent intensity distribution suitable for performing more than one function at the same time, according to an embodiment of the invention. For example, one portion 48 of the broadbrand spectrum 46 comprises a spectral distribution suitable for measuring the amount of a drug present in the tissue 14 (FIG. 1), and another portion 50 of the broadband spectrum 46 comprises a spectral distribution suitable for activating the drug. The combination of the spectrum portions 52 and 54 form the desired output spectrum and desired wavelength intensity dependent distribution of the illumination light 12. The amount of the drug present in the tissue is typically measured by sensing the intensity of the illumination light 12 that is reflected from the drug, or by sensing the intensity of fluorescent light emitted by the drug in response to the illumination light 12. Thus, the progress of the photodynamic therapy may be monitored and the illumination light 12 varied in response to the progress.

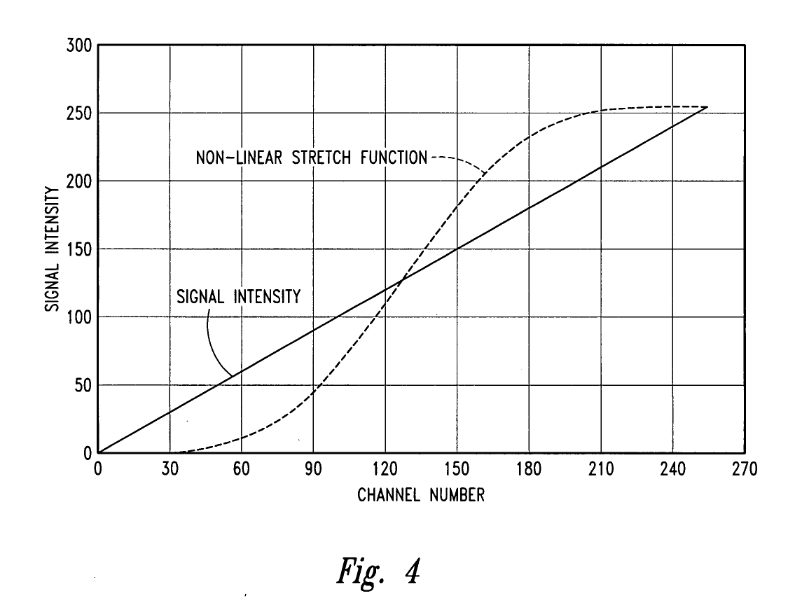

FIG. 4 provides a schematic depiction of the selected spectra 52 and 54 (FIG. 3B) of illumination light 12 (FIG. 1) that the computer-controlled illumination system 12 (FIG. 1) emits sequentially over time, according to an embodiment of the invention. The sequence of selected spectra 52 and 54 may form one sequence for the duration of the phototherapy procedure, photodynamic therapy procedure or diagnostic procedure, or the sequence may be repeated to form a sequence of sequences as desired. Furthermore, the sequence of selected spectra 52 and 54 may include additional, different selected spectra having a desired spectrum output and desired wavelength dependent intensity distribution. Sequencing two or more selected spectra 52 and 54 to form illumination light 12 may be desirable to avoid the individual selected spectra 52 and 54 interfering with each other and thus negatively affecting the ability of each to perform their desired function.

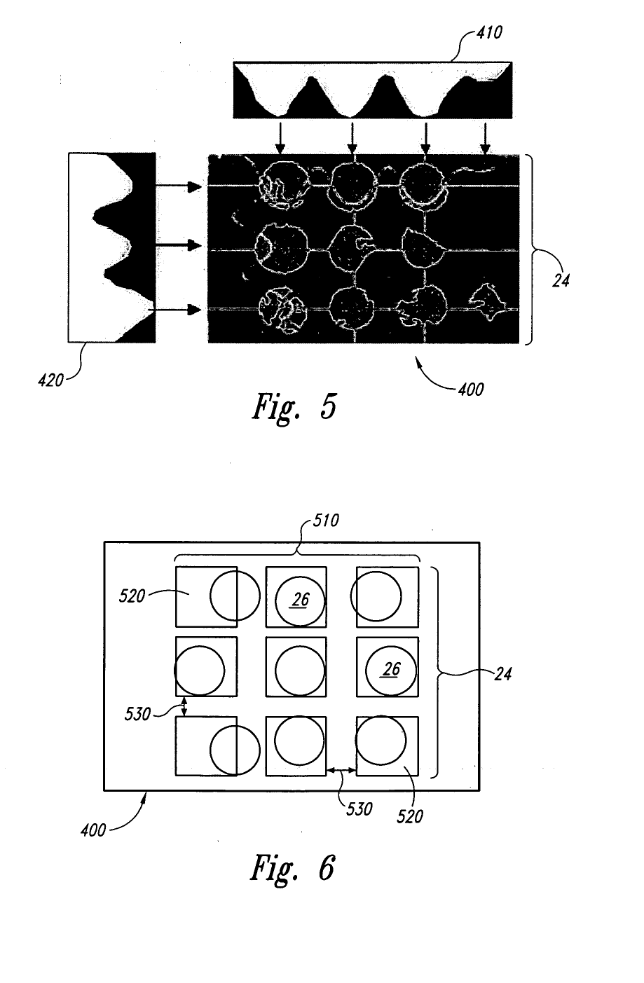

FIG. 5 provides a schematic depiction of selected different exposure intensities and durations of a selected spectrum of illumination light 12 (FIG. 1) that the computer-controlled illumination system 10 (FIG. 1) may emit for phototherapy, photodynamic therapy or diagnosis, according to an embodiment of the invention. The graph 56 represents an exemplary exposure having a high intensity and a short duration, and may be desirable to initiate activation of a drug present in the tissue 14 (FIG. 1) that once initiated no longer requires the selected spectral output and wavelength dependent intensity distribution that the illumination light 12 provides. The graph 58 represents an exemplary exposure having a low intensity and a long duration, and may be desirable to initiate activation of a drug present in the tissue 14 and maintain the drug's activation.

FIG. 6A provides a schematic depiction of an endoscope system 60 comprising the computer-controlled illumination system 10 in FIG. 1, according to an embodiment of the invention. FIG. 6B provides a schematic depiction of a partial view of a distal end of the endoscope system 60. The endoscope system 60 may be used to therapeutically treat tissues not easily accessible. For example, bone, muscle and organs located within a person's body typically can not be reached by illumination light without first exposing them via surgery. In other embodiments, the computer-controlled illumination system 10 may be incorporated in or attachable to surgical microscopes or other optical apparatus such as otoscopes, optical fibers, fiber bundles, liquid light guides and similar devices, to provide illumination light 12 to tissues or other material located in otherwise difficult to reach locations.

The endoscope system 60 comprises a computer-controlled illumination system 10 (FIG. 1) to generate and emit an illumination light (not shown) having a selected spectral output and a selected wavelength dependent intensity distribution, and an endoscope body 62 to direct the illumination light toward the tissue 64. The computer-controlled illumination system 10 is disposed in the embodiment shown at a proximal end of an illumination-light guide 66 (FIG. 6B) of endoscope system 60 and comprises a controller 20. The computer-controlled illumination system 10 emits illumination light that is directed into the illumination-light guide 66. The illumination light is conducted through the endoscope via the illumination light guide 66 to the distal end 68 of the endoscope body 62 where it exits the endoscope system 60 and illuminates the tissue 64.

In some embodiments, a portion of the light emanating from tissue 64 is captured by an objective lens 70 located in the distal end 68 and is directed to form an image of the tissue 64 on image detector 72. Any suitable optical elements may be employed, such as lenses, mirrors, filters for the forming, mixing, imaging, collimating or other conditioning of the light as desired for objective lens 70. Thus, the light emanating from the tissue 64 is passed by the objective lens 70 either by transmitting the light or by reflecting the light or otherwise by acting upon the light. If desired, optical filters and other desired elements can also be provided in the path of the light emanating from the tissue 64, and connected by mirrors, lenses or other optical components.

The image of the sample is transduced by the image detector 72 to create data representative of the image. Image detector 72 may be a charge coupled device (CCD), complementary metal oxide (CMOS) or charge injection device (CID) image detector, or it may be another type of image detector. The image detector 72 is operably connected to an image processing system (not shown) of the controller 20 by the cable 74. The image data from the image detector 72 is transmitted to the controller 20. Transmission of the image data may be effected by electrical signals traveling through conducting wires, optical signals traveling through optical fibers or other optical transmission methods or it may be transmitted by wireless communication devices such as radio waves or other types of wireless devices or networks, or otherwise as desired.

The system controller 20 captures the image data and processes it. With the processed data, the controller 20 may generate a digital image to be displayed so that one can monitor the progress of the phototherapeutic procedure, photodynamic therapeutic procedure or diagnostic procedure. Furthermore, the controller 20 may use the processed data to determine whether to vary the spectral output, the wavelength dependent intensity distribution or both, of the illumination light generated by the computer-controlled illumination system 10, and if so, then to what degree.

In some aspects, the present invention includes light engines and methods related thereto as discussed herein comprising specific, tunable light sources, which can be digital or non-digital. As noted elsewhere herein, one aspect of these systems and methods relates to the ability of the engines to provide finely tuned, variable wavelength ranges that correspond to precisely desired wavelength patterns, such as, for example, noon in Sydney Australia on October 14^{th} under a cloudless sky, or medically useful light of precisely 442 nm. For example, such spectra are created by receiving a dispersed spectrum of light from a typically broad spectrum light source (narrower spectrum light sources can be used for certain embodiments if desired) such that desired wavelengths and wavelength intensities across the spectrum can be selected by the digital light processor to provide the desired intensity distributions of the wavelengths of light. The remaining light from the original light source(s) is then shunted off to a heat sink, light sink or otherwise disposed of (in some instances, the unused light can itself be used as an additional light source, for metering of the emanating light, etc.).

In some aspects, the present invention includes light engines and methods related thereto as discussed herein comprising specific, tunable light sources, which can be digital or non-digital. As noted elsewhere herein, one aspect of these systems and methods relates to the ability of the engines to provide finely tuned, variable wavelength ranges that correspond to precisely desired wavelength patterns, such as, for example, noon in Sydney Australia on October 14^{th} under a cloudless sky, or medically useful light of precisely 442 nm. For example, such spectra are created by receiving a dispersed spectrum of light from a typically broad spectrum light source (narrower spectrum light sources can be used for certain embodiments if desired) such that desired wavelengths and wavelength intensities across the spectrum can be selected by the digital light processor to provide the desired intensity distributions of the wavelengths of light. The remaining light from the original light source(s) is then shunted off to a heat sink, light sink or otherwise disposed of (in some instances, the unused light can itself be used as an additional light source, for metering of the emanating light, etc.).

In the present invention, either or both the light shunted to the heat sink or the light delivered to the target, or other light as desired, is measured. If the light is/includes the light to the light sink, then the measurement can, if desired, include a comparison integration of the measured light with the spectral distribution from the light source to determine the light projected from the light engine. For example, the light from the light sink can be subtracted from the light from the light source to provide by implication the light sent to a target. The light source is then turned up or down, as appropriate, so that as much light as desired is provided to the target, while no more light than desired, and no more power than desired, is emanated from or used by the light source. In the past, it was often undesirable to reduce or increase the power input/output of a given light source because it would change the wavelength profile of the light source. In the present system and methods, this is not an issue because the altered wavelength output of the light source is detected and the digital light processor is modified to adapt thereto so that the light ultimately projected to the target continues to be the desired wavelength intensity distribution.

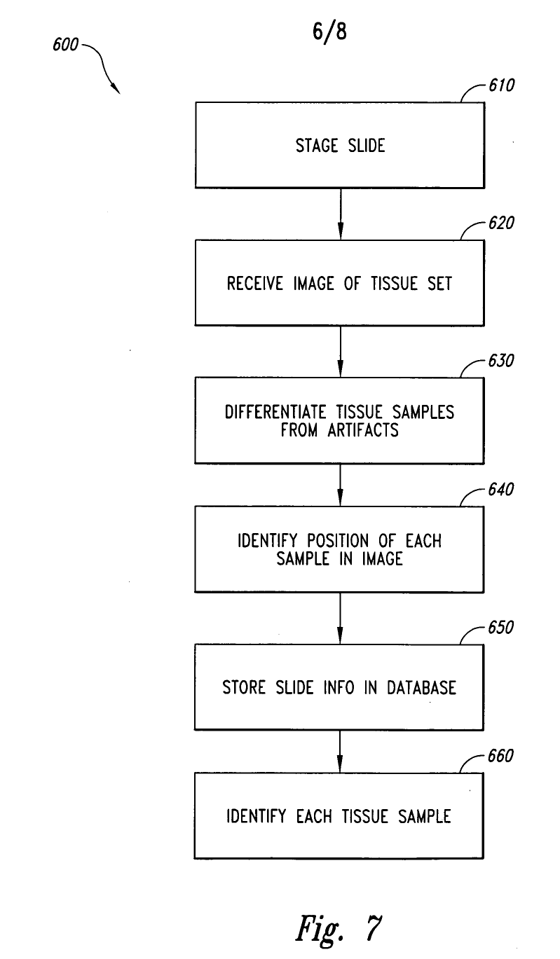

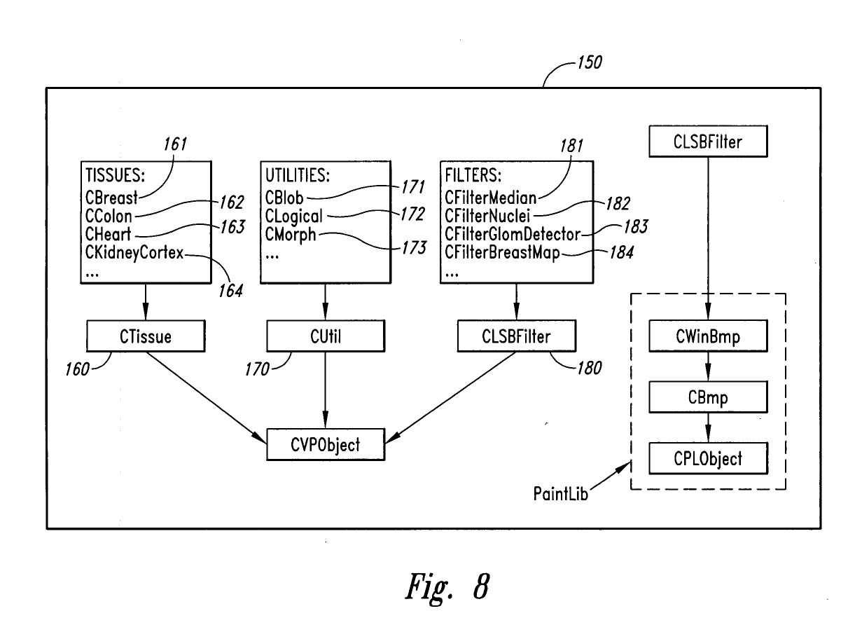

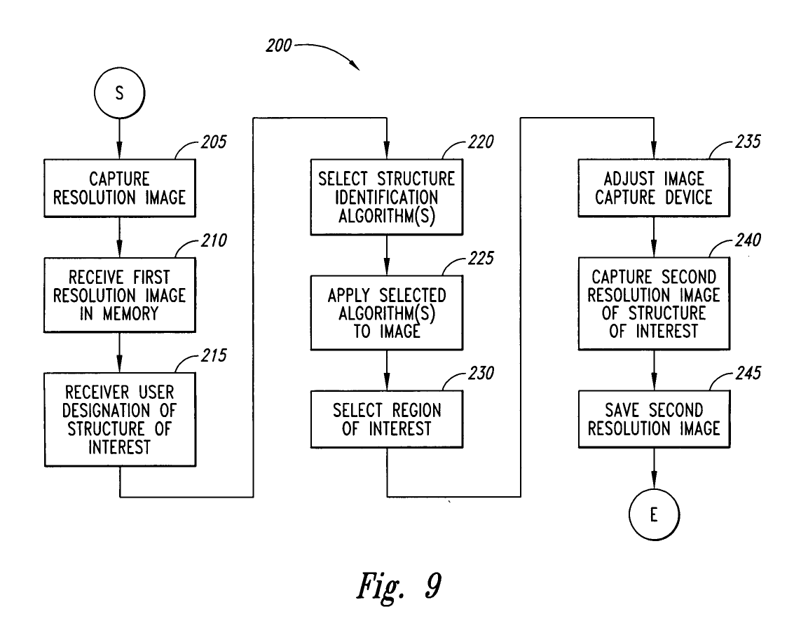

This aspect is depicted in a flow chart, FIG. 7, as follows: Is the wavelength intensity distribution across the spectrum correct? If yes, the proceed with the analysis; if no, then revise the wavelength intensity distribution across the spectrum as desired. Is the intensity target light distribution adequate? If no, then increase power output from light source and repeat. If yes, then proceed to next step. Is there excess light (for example being delivered to the light sink)? If yes, then decrease power to/from the light source. If no, then deem acceptable and leave as is. If power is increased or decreased: Re-check spectral distribution (e.g., of light emanated to target and/or of light from light power source) and if it is changed, reconfigure the digital light processor to adapt to the changed spectral input. If the light engine is changed, then reassess if light source can be turned up or down again. Repeat as necessary.

Some other advantages to the various embodiments herein is that the system is more power friendly, produces less heat, thereby possibly requiring fewer or less robust parts, and in addition should assist in increasing the longevity of various parts of the system due, for example, to the reduced heat generated and the reduced electricity transmitted and the reduced light transmitted. At the same time, this will provide the ability to use particular energy-favorable light sources that might not otherwise be able to be used due to fears over changed spectral distributions due to increased or decreased power output at the light source.

From the foregoing, it will be appreciated that, although specific embodiments of the apparatus and methods have been described herein for purposes of illustration, various modifications may be made without deviating from the spirit and scope of the apparatus and methods. Accordingly, the apparatus and methods include such modifications as well as all permutations and combinations of the subject matter set forth herein and are not limited except as by the appended claims.

1. A computer-controlled illumination system for phototherapy, photodynamic therapy and/or diagnosis, the system comprising:
a tunable light source configured to emit illumination light comprising a variable selected spectral output and a variable selected wavelength dependent intensity distribution;
a sensor configured to detect light emanating from the tunable light source and transmit data representing at least the spectral output and wavelength dependent intensity distribution of the emanating light; and
a controller operably connected to the tunable light source and the sensor, the controller containing computer-implemented programming that is configured to coordinate the tunable light source, the sensor and the processor such that the programming varies the selected spectral output and wavelength dependent intensity distribution of the illumination light to provide a desired spectral output and wavelength dependent intensity distribution for at least one of the following procedures: phototherapy, photodynamic therapy, and diagnosis.

2. The system of claim 1 wherein the illumination light substantially mimics a spectral output and wavelength dependent intensity distribution of at least one of an output energy for disease treatment, an output energy for photodynamic therapy, or an output energy for drug dosimetry.

3. The illumination system of claims 1 wherein the illumination light comprises a fluorescence excitation wavelength.

4. The illumination system of claim 1 wherein the tunable light source includes:
a source of light,
a tunable filter comprising:
a spectrum former able to provide a spectrum from a light beam traveling along a light path from the source of light,
a pixelated spatial light modulator (SLM) located downstream from and optically connected to the spectrum former, the pixelated SLM configured to pass substantially only the selected spectral output and wavelength dependent intensity distribution of the light from the source, the pixelated SLM operably connected to the controller, which contains computer-implemented programming that controls an on/off pattern of pixels in the pixelated SLM to pass substantially only the desired wavelength distributions of illumination light.

5. (canceled)

6. The illumination system of claim 1 wherein the tunable source of light comprises:
a source of light, and,
a tunable filter comprising an acousto-optic tunable filter (AOTF) operably configured to pass substantially only the selected spectral output and wavelength dependent intensity distribution of the light from the light source, the AOTF operably connected to the controller, which contains computer-implemented programming that controls transmission characteristics of the AOTF to pass substantially only the illumination light.

7. (canceled)

8. The illumination system of claim 1 wherein the system further includes a projection system optically connected to and downstream from the tunable filter.

9. The illumination system of claim 1 wherein the system further comprises a heat management system operably connected to the tunable light source to remove undesired energy generated by the tunable light source.

10. (canceled)

11. An endoscope system comprising:
a computer-controlled illumination system comprising:
a tunable light source configured to emit illumination light comprising a variable selected spectral output and a variable wavelength dependent intensity distribution,
a sensor configured to detect light emanating from the tunable light source and transmit a signal representing at least the spectral distribution and wavelength dependent intensity distribution of the emanating light to a processor, and
a controller operably connected to the tunable light source, the sensor and the processor, the controller containing computer-implemented programming that is configured to coordinate the tunable light source, the sensor and the processor such that the programming varies the selected spectral output and wavelength dependent intensity distribution of the illumination light to provide a desired spectral output and wavelength dependent intensity distribution for at least one of the following procedures: phototherapy, photodynamic therapy, and diagnosis; and
an endoscope body comprising a proximal end, a distal end and an illumination light guide, wherein the body is configured to position the distal end proximate to a target tissue, and the illumination guide is optically connectable to the computer-controlled illumination system to emit the illumination light from the distal end.

12. The endoscope system of claim 11 further comprising an image detector operable to receive an image of a target tissue that is generated from light reflected from the target tissue and to transduce the image.

13. The endoscope system of claim 12 further comprising an image processing system operable to acquire the transduced image from the image detector and analyze information in the transduced image to generate data.

14. The illumination system of claim 11 wherein the controller is operably connected to the sensor and contains computer-implemented programming that receives the data from the sensor and uses the data to coordinate the tunable light source and the processor such that the programming varies the selected spectral output and wavelength dependent intensity distribution of the illumination light to enhance the output of the tunable light source.

15. A method for illuminating tissue for at least one of phototherapy, photodynamic therapy or diagnosis, the method comprising:
generating an illumination light containing a desired variable spectral output and a desired variable wavelength dependent intensity distribution from a computer-controlled illumination system comprising:
a tunable light source configured to emit the illumination light,
a detector configured to detect the illumination light and transmit data corresponding to the illumination light, and
a controller operable to vary the a desired variable spectral output and a desired variable wavelength dependent intensity distribution of the illumination light;
detecting the illumination light with the detector; and
directing the illumination light toward a target tissue.

16. (canceled)

17. The method of claim 15 further comprising determining the location of a photodynamic drug in the tissue by generating an illumination light that comprises at least a desired variable spectral output and a desired variable illumination intensity, suitable for causing the photodynamic drug to fluoresce, and then detecting the location of the fluorescence.

18. The method of claim 15 further comprising measuring the amount of a photodynamic drug in the tissue by generating an illumination light that comprises at least a desired variable spectral output and a desired variable illumination intensity suitable for causing the photodynamic drug to do at least one of fluoresce or reflect, and measuring the intensity of the at least one of the fluorescence or reflectance.

19. The method of claim 15 wherein generating the illumination light comprises generating in sequence at least two different variable selected spectral outputs and a variable wavelength dependent intensity distributions, wherein a first of the outputs is suitable for phototherapy and a second of the outputs is suitable for measuring at least one effect of the phototherapy.

20. (canceled)

21. (canceled)

22. The method of claim 15 wherein generating the illumination light comprises generating in sequence at least two different variable selected spectral outputs and a variable wavelength dependent intensity distributions, wherein a first of the outputs is suitable for activating photodynamic therapy and a second of the outputs is suitable for measuring at least one effect of the photodynamic therapy.

23. (canceled)

24. (canceled)

25. The method of claim 15 wherein generating the illumination light comprises generating in sequence at least two different variable selected spectral outputs and a variable wavelength dependent intensity distributions, wherein a first of the outputs is suitable for therapy related to drug dosimetry and a second of the outputs is suitable for measuring at least one effect of the drug dosimetry related to the therapy.

26. The method of claim 19 generating the sequence of illumination light comprises alternating between two spectral distributions of illumination light.

27. The method of claim 15 wherein generating the illumination light comprises:
emitting light from a source of light,
passing the light by a spectrum former optically connected to and downstream from the source of light to provide a spectrum from the light emitted from the source of light, and
passing the spectrum via a pixelated spatial light modulator (SLM) located downstream from and optically connected to the spectrum former, the pixelated SLM configured to pass substantially only the desired spectral output and wavelength dependent intensity distribution of the light from the source to provide the illumination light.

28. The method of claim 27 wherein passing the spectrum via the pixelated SLM comprises reflecting the desired spectral output and wavelength dependent intensity distribution of the light from the source to provide the illumination light.

29. The method of claims 27 wherein passing the spectrum via the pixelated SLM comprises controlling an on/off pattern of pixels in the pixelated SLM with computer-implemented programming contained in a controller, to pass substantially only the desired wavelength distributions of illumination light.

30. The method of claim 15 wherein directing the illumination light toward a tissue comprises projecting the illumination light with a projection system.

31. The method of claim 15 wherein the illumination light comprises infrared light.

32. The method of claim 15 wherein the illumination light comprises ultraviolet light.

33. The method of claim 15 wherein the illumination light consists essentially of light visible to an unaided human eye.

34. The method of claim 15 wherein directing the illumination light toward a tissue comprises passing the illumination light through an illumination light guide of an endoscope.

35. The method of claim 15 further comprising changing the selected spectral output and wavelength dependent intensity distribution of the illumination light in response to the spectral output and wavelength dependent intensity distribution of the illumination light sensed by the sensor.

36. The method of claim 15 further comprising diverting, with a beam splitter, a portion of the illumination light toward the sensor.
